(19) United States
(12) Patent Application Publication
(10) Pub. No.: US 2022/0168588 A1
(43) Pub. Date: Jun. 2, 2022

(54) LOW-LEVEL LIGHT THERAPY FOR RESTORING GUT MICROBIOTA

(71) Applicant: THE GENERAL HOSPITAL CORPORATION, Boston, MA (US)

(72) Inventor: Mei X. Wu, Lexington, MA (US)

(21) Appl. No.: 17/436,714

(22) PCT Filed: Mar. 5, 2020
(86) PCT No.: PCT/US2020/021127

§ 371 Date: Sep. 7, 2021

Related U.S. Application Data
(60) Provisional application No. 62/813,914, filed on Mar. 5, 2019.

(51) Int. Cl. A61N 5/06; A61N 5/067

(52) U.S. Cl. CPC A61N 5/0613; A61N 5/067; A61N 2005/0659; A61N 2005/0652; A61N 2005/0663; A61N 2005/0643

(57) ABSTRACT

Low-level light therapy (LLLT) can be applied to a subject suffering from gut dysbiosis in order to restore the subject's gut microbiota to a state of heath. A subject can be placed proximal to a light source device. The light source device can be configured to apply LLL with wavelengths from the red to infrared part of the spectrum. The LLL can be applied at a power density and for a time sufficient to restore the subjects gut microbiota to a state of health.

### CROSS-REFERENCE TO RELATED APPLICATIONS

This application claims the benefit of U.S. Provisional Application No. 62/813,914, filed Mar. 5, 2019, entitled “LOW-LEVEL LASER THERAPY FOR PROTECTION OF MICROBIOTA UNDER VARIOUS STRESSES”. This provisional application is hereby incorporated by reference in its entirety for all purposes.

### SEQUENCE LISTING

The instant application contains a Sequence Listing which has been submitted in ASCII format via EFS-Web and is hereby incorporated by reference in its entirety. Said ASCII copy, created on Mar. 4, 2020, is named SEQUENCE LISTING.txt and is 3,282 bytes in size.

### TECHNICAL FIELD

The present disclosure relates generally to low-level light therapy (LLLT) and, more specifically, to systems and methods that apply LLLT to a subject suffering dysbiosis of the gut in order to restore the subject's gut microbiota to a state of health.

### BACKGROUND

The human gastrointestinal (GI) tract is the continuous series of organs beginning at the mouth and ending at the anus. Throughout its length the GI tract is colonized by microorganisms of a variety of different species. The collection of bacteria, archaea, and eukarya colonizing the GI tract is termed the “gut microbiota.” The microbiota, especially the gut microbiota, affect metabolism, neurological and cognitive functions, hematopoiesis, inflammation and immunity. An intact and functional gut epithelium maintains a healthy body and gut epithelial homeostasis is maintained by continuous crosstalk between the gut microbiota, immune cells and mucosal barrier.

Despite recent advances in anti-cancer drugs, chemotherapeutic agents that impair cell proliferation remain the standard method of treatment for many types of cancer. However, the use of chemotherapeutic agents results in numerous detrimental side effects due to the combination of systemic administration of chemotherapy and its lack of cellular specificity. For example, chemotherapy is known to perturb the mutualism between microbiota and epithelial cells particularly in the gut, leading to body weight loss and altered susceptibility to certain chemotherapy drugs. A safe, cost-effective way to minimize the side-effects of chemotherapy would be beneficial.

### SUMMARY

The present disclosure relates generally to low-level light therapy (LLLT) and, more specifically, to methods and systems that apply low-level light (LLL) to a subject suffering from gut dysbiosis brought on by a bodily stress in order to restore the subject's gut microbiota to a state of health. Chemotherapy is one bodily stress that can cause dysbiosis of the gut, and it has been discovered that LLLT can restore the mutualism between the microbiota and epithelial cells in the gut, and thus, restore the heath of the gut microbiota.

In one aspect, the present disclosure can include a method that includes placing a subject's abdomen and/or back proximal to a light source device. The subject can be suffering from dysbiosis of the gut caused by a bodily stress. The light source device can be configured to apply LLL at a certain wavelength. The LLL can be applied to the subject's abdomen and/or back at a power density and for a time sufficient to restore the subject's gut microbiota to a state of health.

In another aspect, the present disclosure can include a method that includes applying LLL to a subject's abdomen and/or back at a certain wavelength where the subject has previously undergone chemotherapy and/or radiation therapy. The LLL can be applied at a power density and for a time sufficient to restore the subject's gut microbiota to a state of health.

In a further aspect, the present disclosure can include a light source device configured to apply LLL to a subject's abdomen and/or back. The light source device can include a light source configured to generate a light signal with a wavelength from 600 nm to 1640 nm and a power density of from 0.001-1 W/cm², a processing unit preprogrammed with a time for application of the light signal to the abdomen and/or back, and a power source. The time for application can be sufficient to restore the subject's gut microbiota to a state of health.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a block diagram illustration showing an example of a system that is configured to apply low-level light therapy (LLLT) to a subject suffering from gut dysbiosis to restore the subject's gut microbiota to a state of health in accordance with an aspect of the present disclosure;

FIG. 2 is a process flow diagram illustrating a method for delivering LLLT to a subject suffering from gut dysbiosis that can be employed by the LLL generator of FIG. 1;

FIGS. 3(A)-(C) are images of exemplary light source devices in accordance with an aspect of the present disclosure;

FIGS. 4(A)-(D) are experimental results showing LLLT mitigates cachexia induced by chemotherapy/radiation therapy (CRT) and the beneficial effect is transferable by co-housing; (A) is a graph showing the percent body weight of CRT-mice and LLL-mice (CRT-mice are mice only treated with CRT; LLL-mice are mice first treated with CRT and then treated with LLL) over 20 days following CRT where the CRT-mice and LLL-mice were housed separately; (B) is a graph showing average body weight of the CRT-mice and the LLL-mice 20 days after receiving CRT, naïve indicates mice that were not treated with CRT or LLL; (C) is a graph showing the percent body weight of CRT-mice and LLL-mice over 20 days post-CRT where the CRT-mice and LLL-mice were housed together (***p<0.001 in the presence or absence of LLL, ns=no significance); body weight relative to day 0 was monitored every other day (A and C); and (D) is an image showing the device used to administer LLL or sham light;

FIGS. 5(A)-(E) are experimental results showing that LLLT protects against and/or restores CRT-induced intestinal damage; (A)-(B) provide histopathological images of the small intestines of mice 22 days after receiving CRT where (A) shows the mice only treated with CRT, and (B) shows the mice treated with CRT and LLL; image (C) shows the naïve control mice that were not treated with CRT or LLL (arrows indicate corresponding enlargement in the lower panels); (D) is a graph showing the villa length of the CRT-mice, LLL-mice (CRT-mice are mice only treated with CRT; LLL-mice are mice first treated with CRT and then treated with LLL)), and naïve mice; and (E) is a graph showing the ratio of crypt depth to villus length for CRT-mice, LLL-mice, and naïve mice (**p<0.01 and ***p<0.001 in the presence or absence of LLL or between indicated groups);

FIG. 6 is an graph showing that LLLT displays minimal effect on body weight changes in mice who had previously received antibiotics daily for two weeks before CRT;

FIG. 7 is a graph showing that LLLT-mediated benefits are transferrable (*p<0.05; **p<0.01; and ***p<0.001 in the presence or absence of fecal microbial transplants);

FIGS. 8(A)-(B) are graphs showing that LLLT increases the abundance of certain bacterial phyla; (A) is a graph showing the relative abundance of seven different phyla by qPCR in the fecal samples prepared from LLL-mice and CRT-mice (CRT-mice=control) (*p<0.05; **p<0.01; and ***p<0.001 in the presence or absence of LLL; n=5); (B) is a graph showing the relative abundance of three well-known probiotic bacteria by qPCR in the fecal samples prepared from LLL-mice and CRT-mice (CRT-mice=control) (ns=no significance).

### DETAILED DESCRIPTION

### I. Definitions

Unless otherwise defined, all technical terms used herein have the same meaning as commonly understood by one of ordinary skill in the art to which the present disclosure pertains.

In the context of the present disclosure, the singular forms “a,” “an” and “the” can also include the plural forms, unless the context clearly indicates otherwise.

As used herein, the terms “comprises” and/or “comprising” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

Unless otherwise indicated, all numbers expressing quantities used in the specification and claims are to be understood as being modified in all instances by the term “about.” Accordingly, unless otherwise indicated, the numerical properties set forth in the following specification and claims are approximations that may vary depending on the desired properties sought to be obtained. Notwithstanding that the numerical ranges and parameters setting forth the broad scope of the disclosure are approximations, the numerical values set forth in the specific examples are reported as precisely as possible. Any numerical values, however, inherently contain certain errors necessarily resulting from error found in their respective measurements.

Also herein, where a range of numerical values is provided, it is understood that each intervening value is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

As used herein, the term “low-level light (LLL)” can refer to a procedure that involves exposing at least a portion of a patient's body (e.g., the abdomen or back) to low levels of red and/or near infrared (NIR) light at energy densities that are low compared to other forms of laser therapy (e.g., ablation, cutting, thermal coagulation, etc). As used herein, the term “low-level light therapy (LLLT)” can be used interchangeably with LLL.

As used herein, the term “light source device” can refer to a mechanical implement that can deliver a light signal of LLL to a portion of the subject's body. Examples of the light source devices include a probe, a flexible array device, or the like.

As used herein, the term “light source” can refer to a component of a light source device that delivers one or more lights of different wavelengths. For example, the light source can be a low-level laser source (e.g., a laser light emitting diode (LED)) that generates coherent light. As another example, the light source can be an incoherent light source, such as a traditional LED or light bulb. The wavelength of the light can include a wavelength corresponding to the visible range of the electromagnetic spectrum (e.g., red light). In another example, the wavelength can correspond to the near-infrared or infrared range of the electromagnetic spectrum.

As used herein, the term “therapy” and “treatment” are synonymous and can refer to medical care given to a subject to bring about a change in a medical condition, such as gut dysbiosis.

As used herein, the term “proximal” can refer to a location that is near or at a target. For example, a device that is located proximal to the abdomen and/or back can refer to a device that is located near the abdomen and/or back but is not directly touching the abdomen and/or back (e.g., the device is located above, below, or in front of the abdomen and/or back), or “proximal” can refer to a device that is located at the abdomen and/or back and is directly touching the abdomen and/or back.

As used herein, the term “direct” refers to the absence of intervening elements. For example, a device that directly contacts the abdomen has no intervening elements between the device and the skin of the abdomen.

As used herein, the term “sufficient” refers to an amount adequate enough to satisfy a condition. For example, “a time sufficient to restore the subject's gut microbiota to a state of health” can refer to LLL being applied to the abdomen for a time adequate enough to restore the subject's gut microbiota to a state of health.

As used herein, the terms “subject” and “patient” can be used interchangeably and refer to any warm-blooded organism including, but not limited to, a human being, a pig, a rat, a mouse, a dog, a cat, a goat, a sheep, a horse, a monkey, an ape, a rabbit, a cow, etc.

### II. Overview

Low-level light therapy is commonly used for pain management, to reduce inflammation, and to stimulate photo-biological responses to enhance physiological reactions. The present disclosure relates generally to low-level light therapy (LLLT) and, more specifically, to systems and methods that apply low-level light (LLL) to a subject suffering from gut dysbiosis in order to restore the subject's gut microbiota to a state of health. As used herein, the phrase “restore the subject's gut to a state of health” can mean that (i) at least one aspect of the subject's gut health is improved as compared to the state that the subject's gut health was in before LLL was administered to the subject, and/or (ii) at least one aspect of the subject's gut health is improved as compared to the state that the subject's gut health would be in if no LLL was administered. Aspects of improvement in gut health may include, for example, (i) an increase in abundance of one or more beneficial phyla in the subject's gut, (ii) a decrease in abundance of one or more harmful phyla in the subject's gut, (iii) preserved villus architecture, (iv) restored villus architecture, (v) an increase in villus length, (vi) a decrease in the formation of pyknotic nuclear structures, (vii) preserved crypt depth, and (viii) a decrease in the crypt depth/villus length ratio.

It has been surprisingly found that LLLT can be used to improve gut health in a subject suffering from gut dysbiosis. LLLT provides a safe, non-invasive, and non-pharmacological therapy to patients suffering from the effects of certain bodily stresses that are associated with gut dysbiosis, e.g., chemotherapy induced cachexia. It has been found that applying LLL to a subject suffering from gut dysbiosis can increase the abundance of certain beneficial phylum of bacteria in the subject's gut such as Tenericutes, Verrucomicrobia, and Deferribacteres. The LLLT can be used cumulatively on a subject who is undergoing e.g., multiple rounds of chemotherapy/radiation therapy (CRT). The LLLT can be used alone or in combination with alternative treatments to restore the subject's gut microbiota to a state of health.

While not wishing to be bound by theory, it is generally believed that the beneficial effects of LLL can be ascribed to the ability of LLL to enhance mitochondrial function in mammalian cells. However, bacteria do not have mitochondria. It is possible that certain bacteria may respond to light similar to mitochondria and use light to synthesize ATP because ATP synthase, proton-motive force, and electron transport chain are found within these certain bacteria. Thus, it is possible that LLL can be employed to restore a subject's gut health at least because individual bacterial phyla respond differently to light and have differing abilities to use light to generate ATP.

### III. Restoring Gut Microbiota

The present disclosure provides a method that includes applying LLL to a subject suffering from gut dysbiosis, wherein the gut dysbiosis is caused by a bodily stress, to restore the subject's gut microbiota to a state of health. The term “dysbiosis” can refer to a microbial imbalance inside the body. The human microbiome is made up of a large number of different strains and species of bacteria. The most dominant of which, in a healthy gut, are commensal bacteria. However when the quantity and proportion of commensal bacteria are reduced, harmful strains and pathogens can grow unchecked causing an imbalance in the microbiota which can lead to dysbiosis.

There are a number of known bodily stresses that can cause gut dysbiosis. For example, certain acute bodily stresses can cause gut dysbiosis, including but not limited to, chemotherapy, radiation therapy, diarrhea, inflammatory bowel disease, and cancer immunotherapy. In other instances, certain chronic bodily stresses can cause gut dysbiosis, including but not limited to, metabolic disorders (e.g., obesity, diabetes), neurodegenerative diseases (e.g., pain, depression, anxiety, autism, multiple sclerosis, Huntington's disease, Parkinson's disease, Alzheimer's disease, stroke) allergies, asthma, and autoimmune diseases (e.g., lupus, ulcerative colitis, Crohn's disease).

It has been discovered that LLLT can be used to restore gut health. LLL is a simple, non-invasive, safe, convenient, and cost-effective modality that has been clinically employed for decades for pain relief and other applications.

In one aspect of the present disclosure, low-level light (LLL) can be applied (in one dose or in multiple doses) to at least a portion of a patient's body at energy densities that are low compared to other forms of laser therapy (e.g., ablation, cutting, thermal coagulation, etc.). For example, the LLL energy density can be from 0.001 J/cm² to 50 J/cm². As another example, the LLL energy density can be from 0.001 J/cm² to 20 J/cm². In another example, the LLL energy density can be from 0.001 J/cm² to 3 J/cm². In a further example, the LLL energy density can be from 0.01 J/cm² to 0.5 J/cm². In a specific example, the LLL energy density can be 0.025 J/cm². In certain instances, the energy density can be measured at the middle of the subject's abdomen. In other instances, the energy density can be measured at different locations along the subject's gut.

The LLL used herein, in some examples, can have a wavelength from 600 nm to 1640 nm. In other examples, the LLL can have a wavelength from 600 nm to 1000 nm, 700 nm to 1000 nm, 800 nm to 1000 nm, or 900 nm to 1000 nm. In still other examples, the LLL can have a wavelength from 650 nm to 750 nm or 800 nm to 900 nm. In one particular example, the LLL can have a wavelength of 980 nm. In another particular example, the LLL can have a wavelength of 660 nm. In a further example, the LLL can have a wavelength of 810 nm.

The power density of the LLL can be from 0.001 W/cm² to 1 W/cm². In one example, the power density can be from 0.025 W/cm² to 0.5 W/cm². In certain instances the power density can be 0.1 W/cm² or more. In one instance, the selected or predetermined power density to be delivered to the body can be selected from the range of about 0.01 W/cm² to about 0.15 W/cm², including e.g., 0.015, 0.02, 0.03, 0.04, 0.05, 0.06, 0.07, 0.08, 0.09, 0.1, 0.11, 0.12, 0.13, and 0.14 W/cm², and all values there between. In another instance, the selected or predetermined power density to be delivered to the body can be selected from the range of about 0.2 W/cm² to about 0.5 W/cm², including, e.g., 0.3 W/cm² and 0.4 W/cm², and all values there between. In some instances, higher power densities can be used. To attain subsurface power densities within these ranges in in vivo methods, one skilled in the art would understand that one must take into account attenuation of the energy as it travels through body tissue and fluids from the surface to the target tissue.

In certain aspects, the LLL can be applied in a single dose. In other instances, the LLL can be applied in multiple doses. In some instances, the LLL can be applied multiple times on the same day (e.g., one to five times a day). In further instances, the LLL can be applied over the course of multiple days (e.g., two to ten days). In further instances, the LLL can be applied on consecutive days. In other instances, the LLL can be applied on an intermittent basis, e.g., every other day. In certain instances, the LLL can be applied e.g., once or twice a day for 1-10 days. In one example, the LLL can be applied once a day for four days. In another example, the LLL can be applied once a day for five days. In another example, the LLL can be applied twice a day for four days. In a further example, the LLL can be applied twice a day for five days.

The LLL can be applied to the subject for a period of 10 minutes to 3 hours. In one example, the LLL can be applied for a period of 30 minutes to 2 hours. In one specific instance, the LLL can be applied for a period of 30 minutes. In an additional instance, the LLL can be applied for 1 hour. In another specific instance, the LLL can be applied for a period of 2 hours. The period of time that the LLL is administered to the subject can be based in part on the body mass index (BMI) of the subject. For example, subjects having a lower BMI may only require LLL administration for a period of 30 minutes, whereas subjects with a higher BMI may require that the LLL be administered for a period of 2 or 3 hours.

In some instances the LLL can be applied to at least a portion of a subject's body. One skilled in the art will understand that in each instance the LLL can be applied proximally to the subject's body. In certain instances the LLL can be applied directly to the subject's body. In one aspect, the LLL can be applied to the subject's abdomen and/or back. In one instance, the LLL can be applied to the subject's abdomen. In another instance, the LLL can be applied to the subject's back. In one particular example, the LLL can be applied to the subject's lower back. In another example, the LLL can be applied to the subject's middle back. In yet another instance, the LLL can be applied along the middle of the subject's back. In other instances, the LLL can be applied to the subject's abdomen and the subject's back. In one instance the LLL can be applied to the subject's abdomen and the subject's lower back. In a further instance, the LLL can be applied to the subject's abdomen and the subject's middle back.

In one aspect, applying LLL to a subject suffering from gut dysbiosis can restore the subject's gut microbiota to a state of heath. Restoring the subject's gut to a state of health can mean that (i) at least one aspect of the subject's gut health is improved as compared to the state that the subject's gut health was in before LLL was administered to the subject and/or (ii) at least one aspect of the subject's gut health is improved as compared to the state that the subject's gut health would be in if no LLL was administered. Aspects of improvement in gut health may include, for example, (i) an increase in abundance of one or more beneficial phyla in the subject's gut, (ii) a decrease in abundance of one or more harmful phyla in the subject's gut, (iii) preserved villus architecture, (iv) restored villus architecture, (v) an increase in villus length, (vi) a decrease in the formation of pyknotic nuclear structures, (vii) preserved crypt depth, and (viii) a decrease in the crypt depth/villus length ratio.

In one instance, restoring the subject's gut microbiota to a state of health can include increasing the abundance of one or more of the phyla consisting of Tenericutes, Verrucomicrobia, and Deferribacteres. In another instance, restoring the subject's gut microbiota to a state of health can include increasing the abundance of the Tenericutes and Verrucomicrobia phyla in the subject's gut. In a further instance, restoring the subject's gut microbiota to a state of health can include increasing the abundance of the Tenericutes, Verrucomicrobia, and Deferribacteres phyla in the subject's gut. In another example, restoring the subject's gut microbiota to a state of health can include increasing the abundance of the Tenericutes phyla in the subject's gut. In a further example, restoring the subject's gut microbiota to a state of health can include increasing the abundance of the Verrucomicrobia phyla in the subject's gut. In yet another example, restoring the subject's gut microbiota to a state of health can include increasing the abundance of the Deferribacteres phyla in the subject's gut. In another instance, restoring the subject's gut microbiota to a state of health can include decreasing the abundance of harmful phyla in the subject's gut. In other instances, restoring the subject's gut microbiota to a state of health can include restoring and/or preserving villus architecture. In certain instances, restoring the subject's gut microbiota to a state of health can include preventing the shortening of villus length, and/or preventing the formation of pyknotic nuclear structures, and/or preserving crypt depth.

In some instances, the LLLT can be used in conjunction with other treatments. For example, LLLT can be used in conjunction with other treatments that help restore a healthy microbiota in the gut. For instance, LLLT can be used in conjunction with various diets, supplements, nutritional plans, and probiotic treatments.

In one aspect, the present disclosure includes a method that includes applying LLL to a subject suffering from gut dysbiosis caused by a bodily stress. In one instance, the method can include placing a subject's abdomen and/or back proximal to a light source device wherein the light source device is configured to apply LLL at a certain wavelength. The method can further include applying the LLL to the subject's abdomen and/or back at a power density and for a time sufficient to restore the subject's gut microbiota to a state of health.

In another aspect, the present disclosure includes a method that includes applying LLL to a subject's abdomen and/or back at a certain wavelength wherein the subject has previously undergone chemotherapy and/or radiation therapy. In one instance, the LLL can be applied at a power density and for a time sufficient to restore the subject's gut microbiota to a state of health.

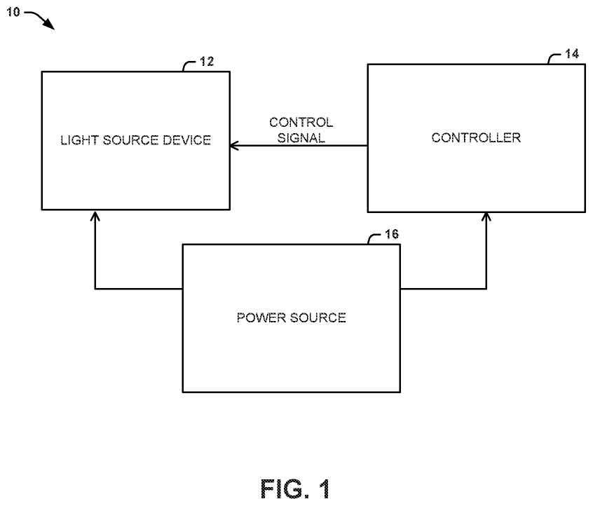

As shown in FIG. 1, application of the LLL can be controlled by a system 10 that can include a light source device 12, a controller 14, and a power source 16. The power source 16 can be configured to supply an operating power to the controller 14 and/or the light source device 12. In some instances, the controller 14 and/or the light source device can be electrically coupled to the power source 16. For example, the power source 16 can be a device that is configured to generate a power signal (e.g., including enough power to power up the controller 14 and/or the light source device), such as a battery power source, a line power source (e.g., a plug), or the like. In other instances, the power source can include at least two power sources (e.g., one to power the light source device 12 and one to power the controller 14).

The controller 14 can be configured to generate and transmit a control signal (e.g., including dosage parameters for LLL) to the light source device 12. The controller 14 can be a computing device (e.g., a general purpose computer, special purpose computer, and/or other programmable data processing apparatus) that can include or be otherwise associated with a non-transitory memory storing instructions (e.g., computer program instructions) that, upon execution by a processor, can create a mechanism for implementing the functions of the controller 14 (e.g., generating and transmitting the control signal to the light source device 12). For example, one or more of the dosage parameters (e.g., time of application) can be pre-set within the controller 14. As another example, one or more of the dosage parameters can be input by a user via a user interface associated with the controller 14.

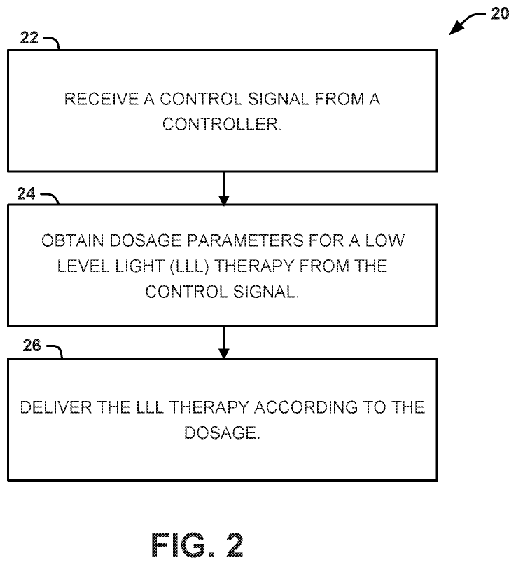

The controller 14 and the light source device 12 can be communicatively coupled (e.g., via a wired connection and/or a wireless connection) to facilitate the transmission of the control signal. An example of a method 20 that the light source device 12 can utilize to apply the LLL according to the control signal is shown in FIG. 2. For purposes of simplicity, the method 20 is shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the method 20. At 22, the light source device 12 can receive the control signal from the controller 14. The control signal can include dosage parameters, including an on time, an off time, a light density, a power (e.g., between 0.001 W and 1 W), a power density e.g., between 0.001 W/cm² and 1 W/cm²), and an output characteristic (pulsed (frequency=1 Hz-500 Hz) or continuous). One skilled in the art would understand that more specific dosage parameters can include those disclosed previously.

At 24, the light source device 12 can obtain the dosage parameters for the LLL from the control signal. At 26, the light source device can deliver the LLL according to the dosage. In some instances, the dosage can include a desired power density (e.g., selected from 0.001 W/cm² to 1 W/cm²) to be delivered to the intestines. In one specific instance, the dosage can include a desired power density of 300-400 mW/cm². To deliver the desired power density into the intestines, a relatively higher surface power density of LLL may be required, depending on subject's skin pigmentation and the depth of the intestines relative to the skin surface. In some instances, super pulsed GaAs lasers can be employed to generate super pulses of LLL with extremely short duration (100 to 300 nanoseconds), that can penetrate into tissue depths of 3 to 13 cm and deeper.

The light source device 12 can be configured to apply LLL to a subject's body (e.g., the whole body or a portion of the body including the abdomen and/or the back). In some instances, the light source device 12 can include a monochromatic laser that radiates light in the red or NIR wavelengths (λ=600 nm-1640 nm). In other instances, the light source device 12 can include a light emitting diode (LED) that radiates light in the red or NIR wavelengths (λ=600 nm-1640 nm). In one particular instance, the light source device 12 can radiate light at a wavelength of 980 nm (NIR).

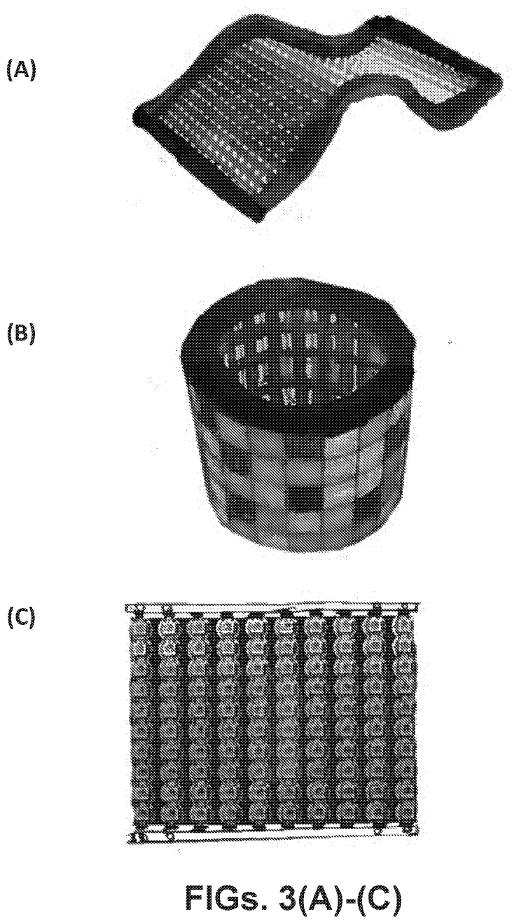

In some instances, the light source device 12 can be configured to apply the LLL to the subject's body according to the control signal. In some instances, the LLL can be applied to a portion of the subject's body (e.g., abdomen and/or back). One skilled in the art will appreciate that any known light source device can be used to apply LLL to at least a portion of the subject's body. The light source device 12 can be configured in any shape that facilitates indirect delivery of LLL to at least a portion of the subject's body or facilitates the direct delivery of LLL to at least a portion of the subject's skin. In one instance the light source device 12 can be a mobile or a stationary device. In another instance, the light source device 12 may be a probe, e.g., a mobile probe or a handheld probe. One example of the light source device 12 that can deliver the LLL to least a portion of the subject's body is a LLL blanket (FIG. 3A). The LLL blanket can be wrapped around the subject's body to deliver the LLL according to the control signal. Another example of the light source device 12 is a LLL vest. The LLL vest can cover at least a portion of the subject's abdomen and/or back to deliver LLL according to the control signal. Yet another example of the light source device 12 is an LLL chair. A subject can sit on the LLL chair, which can deliver the LLL to the subject's back according to the control signal. In another example, an LLL bed can be arranged similarly to a tanning bed to deliver LLL to at least a portion of the subject's body. In a further example, a LLL cylinder can be used to deliver the LLL to least a portion of the subject's body (FIG. 3B). In yet another example, a LED panel can be used to deliver the LLL to least a portion of the subject's body (FIG. 3C).

In one aspect, the present disclosure includes a light source device configured to apply LLL to a subject's abdomen and/or back. The device can include a light source configured to generate a light signal with a wavelength from 600 nm to 1640 nm and a power density of from 0.001-1 W/cm². The light source device can also include a processing unit preprogrammed with a time for application of the light signal to the abdomen and/or back, wherein the time for application is sufficient to restore the subject's gut microbiota to a state of health, and a power source.

In some instances, the light signal can have a wavelength from 600 nm to 1000 nm, 700 nm to 1000 nm, 800 nm to 1000 nm, or 900 nm to 1000 nm. In still other examples, the light signal can have a wavelength from 650 nm to 750 nm or 800 nm to 900 nm. In one particular example, the light signal can have a wavelength of 980 nm. In another particular example, the light signal can have a wavelength of 660 nm. In a further example, the light signal can have a wavelength of 810 nm.

In further instances, the power density of the light signal can be from 0.001 W/cm² to 1 W/cm². In one example, the power density of the light signal can be from 0.025 W/cm² to 0.5 W/cm². In certain instances, the power density of the light signal can be 0.1 W/cm² or more. In one instance, the power density of the light signal can be selected from the range of about 0.01 W/cm² to about 0.15 W/cm², including e.g., 0.015, 0.02, 0.03, 0.04, 0.05, 0.06, 0.07, 0.08, 0.09, 0.1, 0.11, 0.12, 0.13, and 0.14 W/cm², and all values there between. In some instances, higher power densities can be used. For example, power density of the light signal can be selected from the range of about 0.2 W/cm² to about 0.5 W/cm², including, e.g., 0.3 W/cm² and 0.4 W/cm², and all values there between.

In other instances, the time of application of the light signal can be for a period of 10 minutes to 3 hours. In one example, the time of application of the light signal can be for a period of 30 minutes to 1 hour. In one specific instance, the time of application of the light signal can be for a period of 30 minutes. In another specific instance, the time of application of the light signal can be for a period of lhour. In a further specific instance, the time of application of the light signal can be for a period of 2 hours. In another specific instance, the time of application of the light signal can be for a period of 3 hours.

### VI. Experimental

The following examples are shown for the purpose of illustration only and are not intended to limit the scope of the appended claims. This experiment demonstrates the promise of as a non-pharmacological tool for restoring a subject's gut health.

### Example 1

### Methods

Example 1 demonstrates the promise of LLL to mitigate cachexia induced by chemotherapy and/or radiation therapy (CRT).

Study Design

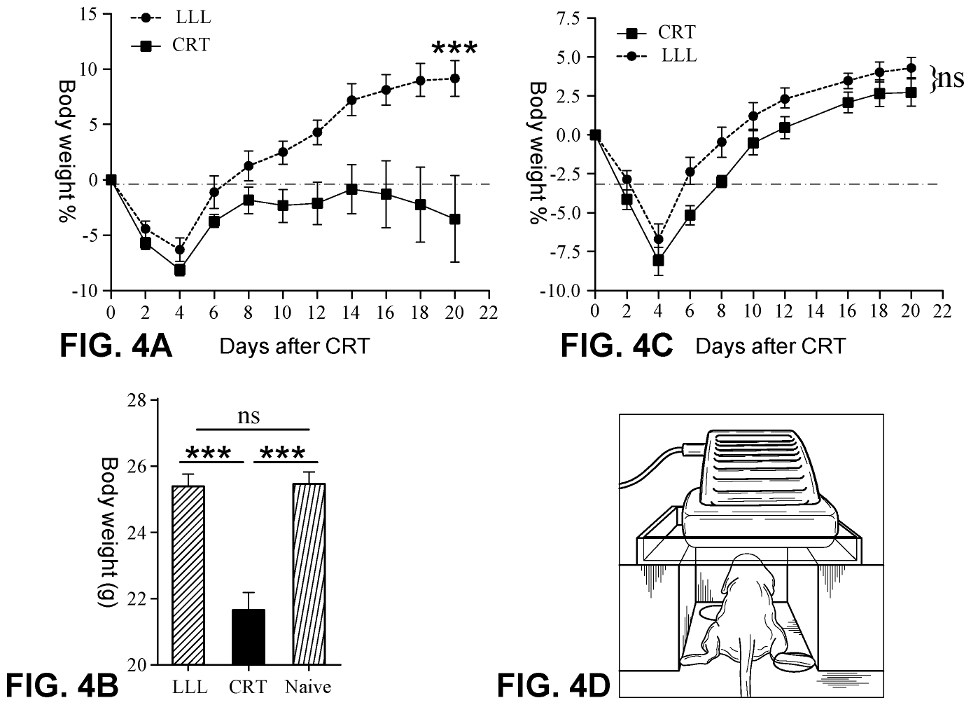

Two individual studies were carried out, each study using fourteen C57BL/6 (B6) female mice at 12 weeks of age. The mice were first intraperitoneally (i.p.) administered a radiosensitizer drug, carboplatin (56.2 mg/kg). Three hours later the mice were subject to 5Gy total body γ-irradiation (TBI) (a cytoablative therapy for cancer and leukemia). In each individual study, LLL at 980 nm was applied to 7 mice and sham light was applied to the other 7 mice. The LLL and the sham light were applied to a shaved, awake mouse noninvasively 3 hours post-TBI as illustrated in FIG. 4D for 123 seconds when the light density was 17.4 mW/cm². The light energy of 980 nm used in each treatment was 0.025 J/cm² measured at the middle of the deceased mouse abdomens. The sham light was a white light with a similar energy intensity. The LLL and sham light treatments were repeated daily for 4 consecutive days. Body weight relative to day 0 was monitored every other day for 20 days, and the average body weight in each group at day 20 was determined. Following CRT and/or CRT/LLL treatment in the first study, the 14 mice from the first study were housed as follows: 4 LLL-mice were housed in a first cage, 3 LLL-mice were housed in a second cage, 4 CRT-mice were housed in a third cage, and 3 CRT-mice were housed in a fourth cage. Following CRT and/or CRT/LLL treatment in the second study, the 14 mice from the second study were housed as follows: 2 LLL-mice and 2-CRT mice were housed in a first cage, 2 LLL-mice and 2-CRT mice were housed in a second cage, 2 LLL-mice and 2-CRT mice were housed in a third cage, and 1 LLL-mouse and 1 CRT-mouse were housed in a fourth cage. The two individual studies were repeated (56 mice were used in total).

### Analysis

CRT caused lower intestine tract damage (FIG. 4), manifested by a precipitous body weight loss with a nadir on day 4 in both LLL-mice and CRT-mice treated with sham light (FIG. 4A). Following the nadir, the body weight in the LLL-mice rose steadily over a two-week period giving rise to a 9% increase in body weight relative to day 0 at the end of experiment (FIG. 4A). Thus, the CRT-mice recovered much faster with LLLT than without it (FIG. 4A). In contrast, despite an initial increase in the body weight for 4 days post-nadir, the body weight remained essentially unaltered thereafter in CRT-mice (FIG. 4A). At the end of the experiment (day 20), LLL-mice weighed similarly as naïve control mice even though the former received CRT and the latter did not (FIG. 4B). The LLL-mice were also much healthier than CRT-mice.

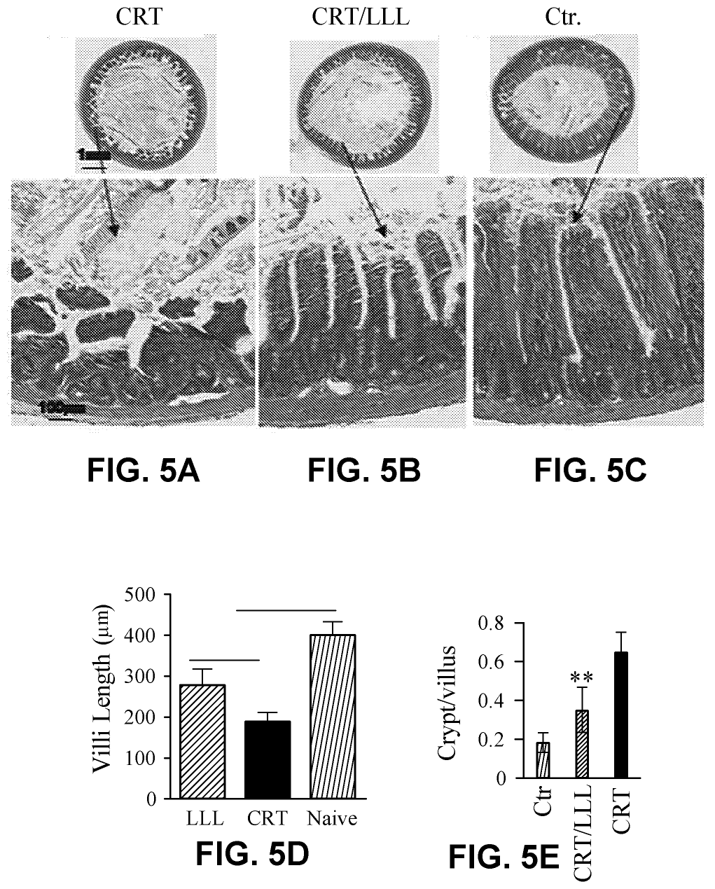

Histopathological studies confirmed that administration of LLL better-preserved villus architecture and villus lengths. In contrast, significant villus loss, destruction, and shortening, and pyknotic nuclear structures in the crypt were found in mice treated with CRT alone (FIG. 5 A vs. B). Morphometric analysis of villus length corroborated highly significant increases in the average length of villi in LLL-mice over CRT-mice, albeit still shorter than those of naïve control mice (FIG. 5D). Similarly, the ratio of the crypt depth/villus length was decreased significantly in LLL-mice over CRT-mice, although the ratio of crypt depth/villus length remained significantly higher than naïve mice (Ctr) (FIG. 5E).

### Co-Housing

Co-housing of LLL-treated mice with CRT sham-treated mice reciprocally affected the healthy status of these two groups of mice. As can be seen in FIG. 4C, the benefit of LLL was blunted when LLL-mice were co-housed with CRT-mice in the same cage. The body weight of LLL-mice increased only 3% when they were co-housed with CRT-mice, decreasing from a 9% increase of body weight at the end of the experiment if they were housed separately from CRT-mice (FIG. 4A vs. C). On the other hand, CRT-mice were healthier in the co-housed cages than those housed separately from LLL-mice, evidenced by an increase of body weight relative to that of day 0 (FIG. 4A vs. C). The observation that co-housing diminishes the difference in body weight changes between the two groups of mice or results in more similarity of their body weight clearly suggests that gut microbiota is involved in both the detriment of CRT and the benefits of LLLT.

### Example 2

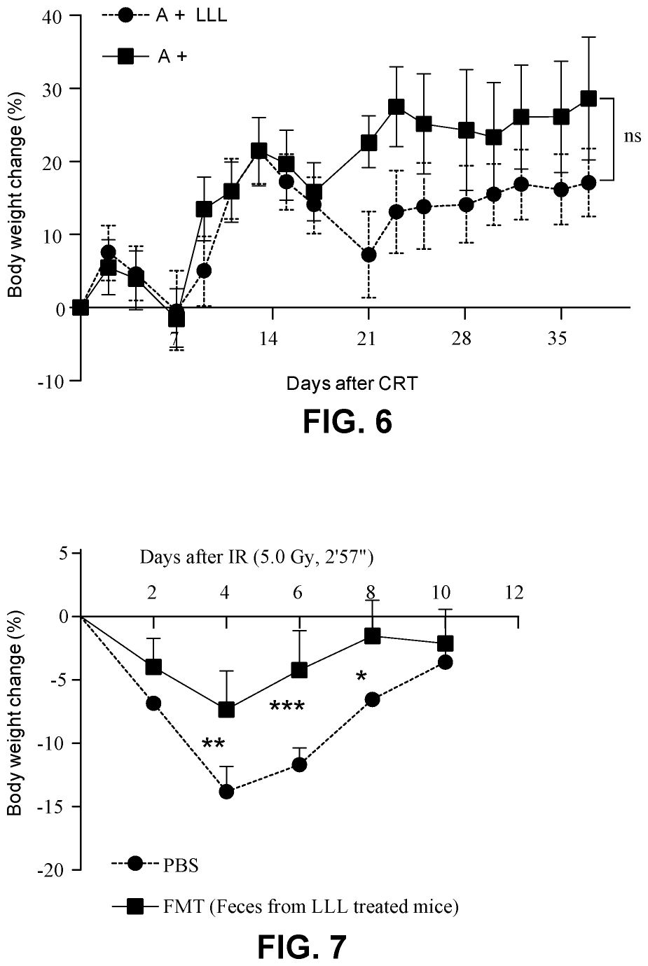

To corroborate the importance of gut microbiota in LLL-mediated protection against gut injury induced by CRT, mice were treated for two weeks with an antibiotic cocktail containing ampicillin (1 mg/mL), streptomycin (5 mg/mL) and vancomycin (0.25 mg/mL) (designated A+ in FIG. 6) in drinking water to eliminate a majority of gut microbes. The antibiotic-containing water administered to the mice was changed out every two days. After two weeks of administering the antibiotics, the bodies of the mice were shaved with the exception of their heads, and the mice were intraperitoneally administered carboplatin (62.5 mg/kg). Three hours later the mice were subject to 5Gy total body γ-irradiation (TBI). The mice were then subjected to either LLL or sham light 3 hours post-TBI (LLL used a laser at 980 nm which delivered 0.025 J/cm² into the intestines). The laser density was adjusted to 17.4 mW/cm² to illuminate the shaved abdomen of the mice for 123 seconds (assuming a penetration rate at 1.3% based on measurements of deceased mice). The LLL and sham light treatments were repeated daily for 4 consecutive days. The body weight of the mice was recorded every other day for 35 days post-CRT. It was found that elimination of gut microbes greatly protected the gut from CRT-induced damage, showing no significant body weight decreases relative to day 0, although a weight loss was noticed on day 7 after CRT (FIG. 6). Importantly, there was no significant difference in body weight changes between LLL and CRT-sham light-treated mice in the “absence” of gut microbiota (FIG. 6). The results suggest that LLL-mediated benefits depend on gut microbiota.

### Example 3

Feces were collected from LLL-treated and the sham-treated CRT-mice during the final week of the experiment described in Example 1 (days 13-20). The fecal samples were weighed and frozen immediately in liquid nitrogen and then stored at −80° C. The frozen fecal pellets were resuspended at a concentration of 100 mg/1.2 mL sterile phosphate-buffered saline (PBS) under anaerobic conditions, homogenized, and filtered through a 70 μm strainer. The recipient mice were irradiated with 5 Gy TBI as described in Example 1 and gavaged with 400 μL of the resultant fecal suspension 3-5 hours after irradiation. The fecal suspension (400 μL) was gavaged once a day for additional four days. As can be seen in FIG. 7, transfer of fecal microbiota prepared from LLL-mice significantly alleviated body weight loss as compared to PBS controls. On the contrary, transfer of the fecal suspension prepared from the sham-treated CRT-mice aggravated the body weight loss and several mice were sacrificed on day 4 due to a body weight loss of more than 20%. The results suggest that LLL favors probiotic microbes under stress.

### Example 4

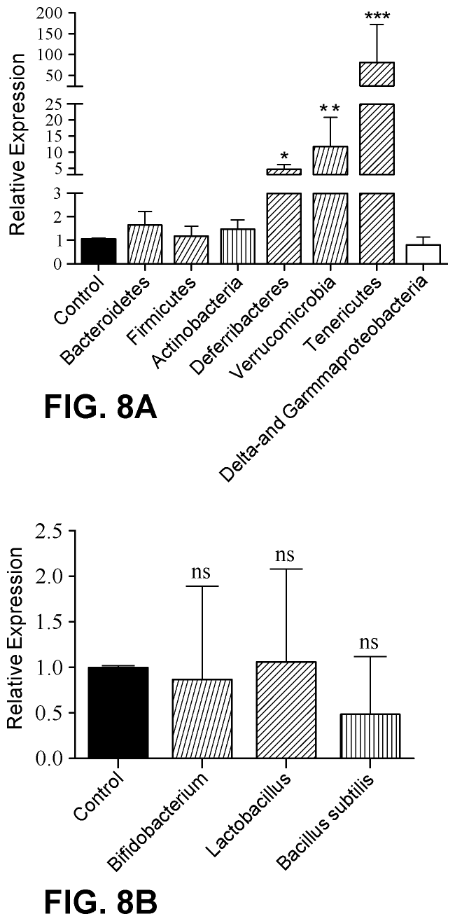

The effect of LLLT on the abundance of specific phyla in the gut microbiota was investigated. To this end, the total DNA of fecal samples collected from LLL-mice and CRT-treated mice, as described in Example 1, were extracted using a fecal genomic DNA extraction kit according to the manufacturer's instructions. Relative expression of seven different groups of bacteria at phylum level (Bacteroidetes, Firmicutes, Actinobacteria, Deferribacteres, Verrucomicrobia, Tenericutes, Delta- and Gammaproteobacteria) was investigated by qPCR. The primers for each phylum are listed in Table 1. An increase of Tenericutes, Verrucomicrobia, and Deferribacteres by 50-fold, 15-fold or 5-fold, respectively was found in LLL-mice as compared with CRT-mice (FIG. 8A, CRT-mice=control). No significant differences in the abundance of Bacteroidetes, Firmicutes, Actinobacteria or δ- and γ-proteobacteria in the presence or absence of LLL were seen. Three-well known probiotic bacteria, specifically Bifidobacterium spp. belonging to the phylum of Actinobacteria, and Lactobacillus spp. and Bacillus subtilis both within the phylum of Firmicutes, were not affected by LLL at a genus level when evaluated by qPCR (FIG. 8B, CRT-mice=control). These data suggest that probiotic bacteria other than Bifidobacterium spp., Lactobacillus spp. and Bacillus subtilis are involved in achieving the LLLT results described in the present disclosure.

TABLE 1

| Genes | Forward (5′-3′) | Reverse (5′-3′) |
|---|---|---|
| Bacteroidetes | GTTTAATTCGATGAT | TTAASCCGACACCTCA |
| | ACGCGAG | CGG |
| | (SEQ ID NO. 1) | (SEQ ID NO. 9) |
| Firmicutes | GGAGYATGTGGTTTA | AGCTGACGACAACCAT |
| | ATTCGAAGCA | GCAC |
| | (SEQ ID NO. 2) | (SEQ ID NO. 10) |
| Actinobacteria | TGTAGCGGTGGAATG | AATTAAGCCACATGCT |
| | CGC | CCGCT |
| | (SEQ ID NO. 3) | (SEQ ID NO. 11) |
| Deferribacteres | CTATTTCCAGTTGCT | GAGHTGCTTCCCTCTG |
| | AACGG | ATTATG |
| | (SEQ ID NO. 4) | (SEQ ID NO. 12) |
| Verrucomicrobia | TCAKGTCAGTATGGC | CAGTTTTYAGGATTTC |
| | CCTTAT | CTCCGCC |
| | (SEQ ID NO. 5) | (SEQ ID NO. 13) |
| Tenericutes | ATGTGTAGCGGTAAA | CMTACTTGCGTACGTA |
| | ATGCGTAA | CTACT |
| | (SEQ ID NO. 6) | (SEQ ID NO. 14) |
| Delta- | GCTAACGCATTAAGT | GCCATGCRGCACCTGT |
| proteobacteria | RYCCCG | CT |
| | (SEQ ID NO. 7) | (SEQ ID NO. 15) |
| Gamma- | AAACTCAAAKGAATT | CTCACRRCACGAGCTG |
| proteobacteria | GACGG | AC |
| | (SEQ ID NO. 8) | (SEQ ID NO. 16) |
| Control | | |

From the above description, those skilled in the art will perceive improvements, changes and modifications. Such improvements, changes and modifications are within the skill of one in the art and are intended to be covered by the appended claims. All patents, patent applications, and publications cited herein are incorporated by reference in their entirety.

The following is claimed:

1. A method comprising:
placing a subject's abdomen and/or back proximal to a light source device wherein the light source device is configured to apply low-level light (LLL) at a certain wavelength, and wherein the subject is suffering from dysbiosis of the gut caused by a bodily stress; and
applying LLL to the subject's abdomen and/or back at a power density and for a time sufficient to restore the subject's gut microbiota to a state of health.

2. The method of claim 1, wherein the certain wavelength is from 600-1640 nm.

3. The method of claim 1, wherein the certain wavelength is selected from 660, 810, and 980 nm.

4. The method of claim 1, wherein the certain wavelength comprises 980 nm.

5. The method of claim 1, wherein the power density is from 0.001-1 W/cm².

6. The method of claim 1, wherein the LLL energy density at middle of the subject's abdomen is from 0.001 J/cm² to 50 J/cm².

7. The method of claim 1, wherein the LLL energy density at middle of the subject's abdomen is from 0.001 J/cm² to 3 J/cm².

8. The method of claim 1, wherein the LLL is applied for a period of 30 minutes to 2 hours.

9. The method of claim 1, wherein the bodily stress is chemotherapy and/or radiation therapy.

10. The method of claim 1, wherein the bodily stress is selected from diarrhea, inflammatory bowel disease, cancer immunotherapy, obesity, diabetes, lupus; irritable bowel syndrome, ulcerative colitis, Crohn's disease, pain, depression, anxiety, autism, Alzheimer's disease, Parkinson's disease, multiple sclerosis, Huntington's disease, stroke, allergies, and asthma.

11. The method of claim 1, wherein restoring the subject's gut microbiota to a state of health comprises increasing the abundance of one or more of the phyla consisting of Tenericutes, Verrucomicrobia, and Deferribacteres in the subject's gut.

12. The method of claim 1, wherein restoring the subject's gut microbiota to a state of health comprises increasing the abundance of the Tenericutes phyla in the subject's gut.

13. The method of claim 1, wherein restoring the subject's gut microbiota to a state of health comprises increasing the abundance of the Verrucomicrobia phyla in the subject's gut.

14. The method of claim 1, wherein restoring the subject's gut microbiota to a state of health comprises increasing the abundance of the Deferribacteres phyla in the subject's gut.

15. The method of claim 1, wherein the light source device comprises an array of light emitting diodes (LEDs) or a laser.

16. The method of claim 1, wherein the light source device comprises a blanket, a cylinder, or a LED panel.

17. A method comprising:
applying low-level light (LLL) to a subject's abdomen at a certain wavelength wherein the subject has previously undergone chemotherapy and/or radiation therapy, and applying the LLL at a power density and for a time sufficient to restore the subject's gut microbiota to a state of health.

18. A light source device configured to apply low-level light to a subject's abdomen and/or back comprising:
a light source configured to generate a light signal with a wavelength from 600 nm to 1640 nm and a power density of from 0.001-1 W/cm²;
a processing unit preprogrammed with a time for application of the light signal to the abdomen and/or back, wherein the time for application is sufficient to restore the subject's gut microbiota to a state of health; and
a power source.

19. The light source device of claim 18, wherein the time for application of the light signal is from 30 minutes to 3 hours.

20. The light source device of claim 18, wherein the certain wavelength comprises 980 nm.


SEQUENCE LISTING (16 sequences)

SEQ ID NO: 1 - DNA, 22 bp, Artificial Sequence
  SYNTHETIC CONSTRUCT
gtttaattcgatgatacgcgag

SEQ ID NO: 2 - DNA, 25 bp, Artificial Sequence
  SYNTHETIC CONSTRUCT
ggagyatgtggtttaattcgaagca

SEQ ID NO: 3 - DNA, 18 bp, Artificial Sequence
  SYNTHETIC CONSTRUCT
tgtagcggtggaatgcgc

SEQ ID NO: 4 - DNA, 20 bp, Artificial Sequence
  SYNTHETIC CONSTRUCT
ctatttccagttgctaacgg

SEQ ID NO: 5 - DNA, 21 bp, Artificial Sequence
  SYNTHETIC CONSTRUCT
tcakgtcagtatggcccttat

SEQ ID NO: 6 - DNA, 23 bp, Artificial Sequence
  SYNTHETIC CONSTRUCT
atgtgtagcggtaaaatgcgtaa

SEQ ID NO: 7 - DNA, 21 bp, Artificial Sequence
  SYNTHETIC CONSTRUCT
gctaacgcattaagtrycccg

SEQ ID NO: 8 - DNA, 20 bp, Artificial Sequence
  SYNTHETIC CONSTRUCT
aaactcaaakgaattgacgg

SEQ ID NO: 9 - DNA, 19 bp, Artificial Sequence
  SYNTHETIC CONSTRUCT
ttaasccgacacctcacgg

SEQ ID NO: 10 - DNA, 20 bp, Artificial Sequence
  SYNTHETIC CONSTRUCT
agctgacgacaaccatgcac

SEQ ID NO: 11 - DNA, 21 bp, Artificial Sequence
  SYNTHETIC CONSTRUCT
aattaagccacatgctccgct

SEQ ID NO: 12 - DNA, 22 bp, Artificial Sequence
  SYNTHETIC CONSTRUCT
gaghtgcttccctctgattatg

SEQ ID NO: 13 - DNA, 23 bp, Artificial Sequence
  SYNTHETIC CONSTRUCT
cagttttyaggatttcctccgcc

SEQ ID NO: 14 - DNA, 21 bp, Artificial Sequence
  SYNTHETIC CONSTRUCT
cmtacttgcgtacgtactact

SEQ ID NO: 15 - DNA, 18 bp, Artificial Sequence
  SYNTHETIC CONSTRUCT
gccatgcrgcacctgtct

SEQ ID NO: 16 - DNA, 18 bp, Artificial Sequence
  SYNTHETIC CONSTRUCT
ctcacrrcacgagctgac